(19) United States
(12) Patent Application Publication
(10) Pub. No.: US 2024/0299464 A1
(43) Pub. Date: Sep. 12, 2024

(54) BOTANICAL COMPOUNDS, METHODS, AND PROCEDURES FOR MEDICINAL TREATMENT RELATED TO IMMUNOINFLAMMATION, NEUROINFLAMMATION, DEPRESSION, MEMORY, FOCUS, AND NERVE DEGENERATION

(71) Applicant: Minh H. Nguyen, Austin, TX (US)

(72) Inventor: Minh H. Nguyen, Austin, TX (US)

(21) Appl. No.: 18/443,193

(22) Filed: Feb. 15, 2024

Related U.S. Application Data
(60) Provisional application No. 63/445,750, filed on Feb. 15, 2023.

(51) Int. Cl. A61K 35/644; A61K 36/258; A61K 36/537; A61K 36/67; A61K 36/899; A61K 36/9068; A61P 25/22; A61P 37/04

(52) U.S. Cl. CPC A61K 35/644; A61K 36/258; A61K 36/537; A61K 36/67; A61K 36/899; A61K 36/9068; A61P 25/22; A61P 37/04

(57) ABSTRACT

The disclosure concerns methods for the inhibiting and treating of immune inflammation such as neuroinflammation, depression, anxiety, cognitive decline, nerve degeneration or the effect of aging, neurological disease, cognitive function, mental disorder, depression, anxiety disorders, stress, respiratory inflammation, post-nasal drip, sinus infections, chronic cough, infections, bacterial infection, viral infection, fungal infection, comprising administering a composition comprising a combination of herbal ingredients.

### CROSS-REFERENCE TO RELATED APPLICATIONS

This application claims benefit of U.S. Provisional Patent Application No. 63/445,750 filed Feb. 15, 2023. The contents of which are incorporated herein by reference in its entirety.

### TECHNICAL FIELD

Embodiments of the present disclosure relate to the field of treatment of immunoinflammation, neuroinflammation, depression, cognitive decline, and nerve degeneration.

### BACKGROUND

The present disclosure relates to a botanical composition and methods for preventing or treating immunoinflammation, such as neurological diseases, depression, anxiety, cognitive decline, nerve degeneration, and aging.

The Central Nervous System (CNS) was once thought to be protected from peripheral immune infiltration. However, recent evidence indicates that the immune system of the CNS is closely connected to peripheral immune responses. Defective communication between the immune and nervous systems is now seen as a common feature in several CNS disorders including neurodevelopmental, neurodegenerative, and neuro-immunological diseases. Multiple mediators and mechanisms have been implicated in the interaction between the CNS and the periphery. The cross-communication between the immune components of the peripheral and CNS systems, such as activated CNS microglia and astrocytes, pro-inflammatory periphery monocytes/macrophages and T lymphocytes, and infiltrated monocytes/macrophages and T lymphocytes, as well as the immunoreactive molecules they release, is closely linked to CNS homeostasis, disease onset and progression.

Neuroinflammation by innate immune cells is defined by the presence of pro-inflammatory cytokines (such as IL-1β, IL-6, and TNFα), chemokines (such as CCL2, CCL5, CXCL1), secondary inflammatory mediators like nitric oxide (NO) and prostaglandins (PGs), and reactive oxygen species (ROS) in inflamed cells and tissues (2). Chronic neuroinflammatory conditions can result in the development of psychiatric diseases. In fact, major depression, anxiety, neurological diseases, and related disorders are linked to a dysregulated immune response, as evidenced by the abnormal levels of pro- and anti-inflammatory cytokines in affected patients. (2). Furthermore, Shen, et al. summarizes how traditional Chinese medicine can assist in the regulation of telomere and telomerase. (4)

The cyclooxygenase (COX) enzymes are comprised of two isoforms, COX-1 and COX-2, which are encoded by distinct genes and exhibit 60% homology in their amino acid sequences. COX-1, the constitutively expressed isoform, is responsible for maintaining normal physiological functions such as gastric protection, modulation of platelet function, and renal homeostasis. In contrast, COX-2 is an inducible isoform that is rapidly increased by pro-inflammatory stimuli such as cytokines, tumor promoters, growth factors, bacterial substances, and thrombin. Both isoforms have been found to be constitutively expressed in the brain, with COX-1 expressed by microglia and perivascular cells and COX-2 identified in post-synaptic dendrites and excitatory terminals in the cortex, hippocampus, and amygdala. This underscores the important neurological functions of COX-2 in the central nervous system, including synaptic activity, long-term potentiation, long-term depression, and memory consolidation. Targeting neuroinflammation by inhibiting pro-inflammatory response of peripheral monocytes/macrophages, such as through the use of non-steroidal anti-inflammatory drugs (NSAIDs) which inhibit COX-1 and COX-2 enzymes, may present a promising future therapeutic strategy to reduce the onset of psychiatric disorders. FIG. 1 shows an example of NSAIDs inhibiting COX-1 and COX-2 enzymes, while FIG. 2 displays some functional differences between the COX-1 and COX isoforms.

The cyclooxygenase enzymes play a role in producing prostaglandins involved in inflammation. Inhibition of COX-2 can impact the serotonergic system in the central nervous system (CNS) both directly and indirectly through its effects on immune processes. A study conducted on rats showed that administration of rofecoxib, a selective COX-2 inhibitor, led to increased serotonin levels in the frontal and temporoparietal cortices. Chronic administration of COX-2 inhibitors has been found to decrease cytokine levels and alter the behavior of individuals with psychiatric disorders, suggesting a potential antidepressant effect. (1).

This disclosure presents compositions for inhibiting and treating inflammatory diseases with broad applications. Inflammation is characterized by an increase in the oxygenation of arachidonic acid, which is metabolized through the cyclooxygenase (COX) and 5-lipoxygenase (5-LO) pathways to produce prostaglandins and leukotrienes, respectively. These substances act as mediators of inflammation, leading to increased interest in therapies aimed at inhibiting COX and/or lipoxygenase activities. See, for example, U.S. Pat. No. 6,391,346.

The relationship between persistent (chronic) infections and the development of psychiatric disorders has been widely explored in research. Elevated levels of pro-inflammatory cytokines, such as IL-1, IL-6, TNF-alpha, and C-reactive protein, have been observed in individuals with major depression. Similarly, increased cytokine levels, particularly IL-6, have been reported in schizophrenia and confirmed through meta-analyses. Microglia, key players in inflammatory processes, have been shown to be activated in both depression and schizophrenia through positron emission tomography studies. This has led to the consideration of immunomodulation as a potential therapeutic approach for major depression and schizophrenia. The COX-2 inhibitor celecoxib has demonstrated efficacy in treating major depression, as evidenced by positive results in both single studies and meta-analyses. Celecoxib has also been studied in early stages of schizophrenia and has shown promising results. (1).

### Anti-TNF

The cytokine Tumor Necrosis Factor-alpha (TNF-α) plays a crucial role in the activation of both innate and adaptive immune responses and is a key mediator of inflammation. Infliximab, an anti-TNF-α antibody, works by blocking the interaction of TNF-α with its receptors on the cell surface and exerting an anti-inflammatory effect. Initially developed for the treatment of inflammatory joint disorders and psoriasis, infliximab has been shown to significantly improve symptoms of depression in patients with psoriasis. (1).

The gut microbiome holds significant significance for human physiology. The composition of an individual's gut microbiota is unique and is impacted by genetics, development, and geographical location. With a large population of primarily anaerobic bacteria, estimated at 1018 microorganisms, the gut microbiome plays a crucial role in bowel function, food digestion, and nutrient absorption. There exists a bidirectional relationship between the brain and gut, which can greatly impact stress, anxiety, depression, and cognitive function. The healthy gut microflora influences brain signals through pathways related to neurogenesis, neural transmission, microglial activation, and behavioral control in both stable and stressful conditions. Any alteration in gut microbiome composition can activate production of microbial lipopolysaccharides, inducing an inflammatory response and sending signals to the vagus nerve that affects the hypothalamic-pituitary-adrenal axis and behavior. Additionally, the gut's inflammation may lead to neuroinflammation and activate the kynurenine pathway. Various factors, such as cytokines, infectious substances, antibiotics, and vagal sensory fibers, among others, can also send signals to the brain regarding the state of the gut. The hypothalamic-pituitary-adrenal axis also plays a role in regulating microbiome diversity and nutrient availability, contributing to the development of depression. (3).

Major depression is a psychiatric disorder characterized by symptoms such as intense sadness, mental sluggishness, concentration difficulties, pessimistic thoughts, agitation, self-criticism, and physical changes. In severe cases, symptoms can include insomnia or excessive sleeping, changes in appetite and weight, decreased energy levels and libido, and disruptions in normal circadian rhythms of activity, body temperature, and endocrine functions. The etiology of depression is complex and can involve a combination of genetic, familial, biochemical, physical, social, and psychological factors. The disorder affects people of all backgrounds and both sexes, with a higher incidence observed in women, particularly in adolescent girls., See, for example, U.S. Pat. No. 2004/0180104.

The management of depressive disorders often involves long-term administration of antidepressant medication. Presently, there are various classes of antidepressants available for treating depression, including tricyclic antidepressants (e.g., imipramine, clomipramine, trimipramine), tetracyclic antidepressants (e.g., maprotiline, mianserin), triazolopyridines (e.g., trazodone), and benzoxazinone-based selective serotonin reuptake inhibitors (e.g., fluvoxamine).

However, the use of tricyclic or similar cyclic antidepressants can result in adverse effects, including anticholinergic effects (such as dry mouth, blurred near vision, constipation, and dysuria), antihistamine effects (such as weight gain and sedation), antiadrenergic effects (such as postural hypotension, vertigo, and dizziness), and cardio toxicity or acute poisoning in the case of excessive intake. Additionally, selective serotonin reuptake inhibitors have been linked to the risk of inducing serotonin syndrome. As a result, there is a pressing need for antidepressants with fewer side effects.

St. John's Wort has been reported to be an effective treatment for depression, as noted in a study published in the British Medical Journal (K. Lindle et al., 1996). The dried aerial parts of the Hypericum perforatum plant, commonly known as St. John's Wort, have been utilized for medicinal purposes in Europe for wound healing and neuralgia. Flavonoids such as hyperoside and isoquercitrin found in St. John's Wort have demonstrated an antidepressant effect (V. Butterweck et al., 2000). However, St. John's Wort also contains hypericin, a dimer structure of anthraquinone, which is associated with serious side effects such as photosensitivity (B. Geoffrey, 1998). The compound hyperforin in St. John's Wort has been shown to affect the pharmacokinetics of drugs such as cyclosporin through induction of drug metabolizing enzymes (F.-B. Adriane, 2000; L. B. Moore et al., 2000). Despite its potential as an antidepressant, the efficacy of St. John's Wort is limited, and there remains a need for the development of herbal drugs with improved anti-depression properties.

Resistant depression often presents with a variety of comorbid health problems such as gastro-intestinal, endocrine, and lipid disorders and gastrointestinal which imply complex physiological and gut pro-inflammatory mechanisms may be at work in terms of disease development and response to treatment.

There remains a need for the developments of improved efficacious therapeutics options for the treatment of depression and related disorders.

### SUMMARY

One method of the disclosure is directed to inhibiting or treating conditions such as immunoinflammation and neurological diseases such as, but not limited to, depression, anxiety, cognitive decline, and aging (cardiovascular, qi and DNA telomeres).

In some methods of the disclosure, the composition may comprise extract from the family of Araliaceae, Zingiberaceae, Poaceae, Lamiaceae, Plantaginaceae, Asteraceae, Piperaceae, Rhytismataceae, Ginkgoaceae, Cucurbitaceae, and/or Fabaceae.

The pharmaceutically acceptable compositions may comprise plants belonging to the family of Araliaceae, Zingiberaceae, Poaceae, Lamiaceae, Plantaginaceae, Asteraceae, Piperaceae, Rhytismataceae, Ginkgoaceae, Cucurbitaceae, and/or Fabaceae.

In one embodiment of the disclosure, the extract may be in tablet, capsule, liquid, gel, gummies, nasal and oral sprays. The extract may be applied in tea, cosmetics, supplements, medicines, or pharmaceutical compositions.

The disclosure is also directed to methods of treating symptoms of immunoinflammation, including immune inflammations, neurological diseases such as, but not limited to, depression, anxiety, cognitive decline, and aging (cardiovascular, qi and DNA telomeres).

In another embodiment, the disclosure is directed to a method for preparing compositions comprising extract of one or more of the following: Panax quinquefolium, Zingiber officinale Roscoe, Curcuma longa, Cymbopogon, Jerusalem sage (Phlomis fruticosa), Common sage (Salvia officinalis), Greek sage (Salvia fruticosa), Grage sage (Salvia melissordora), Grape fruit sage (Salvia gesneriiflora), South African sage (Salvia lanceolata), Graham's sage (Salvia microphylla), white sage (Salvia apiana), California sage (Salvia columbariae), Autum sage (Salvia greggii), Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (plectranthus amboinicus), Scoparia Dulcis, Thymus vulgaris, Lavendula, Matricaria chamomilla, Piper nigrum, Propolis, Silybum marianum, Ginkgo biloba, Stevia rebaudiana, Siraiti grosvenorii, Stigma maydis, Imperata cylindrica, Mentha piperita, Glycyrrhiza glabra, and honey for treating immunoinflammation, neurological diseases (depression, anxiety, improve memories impairment and focus, aging (cardio, qi and DNA telomeres).

### DESCRIPTION OF THE DRAWINGS

The invention is illustrated by the following non-limiting figures.

FIG. 1 presents an example of NSAIDs inhibiting COX-1 and COX-2 enzymes.

FIG. 2 presents some functional differences between COX-1 and COX-2 isoforms.

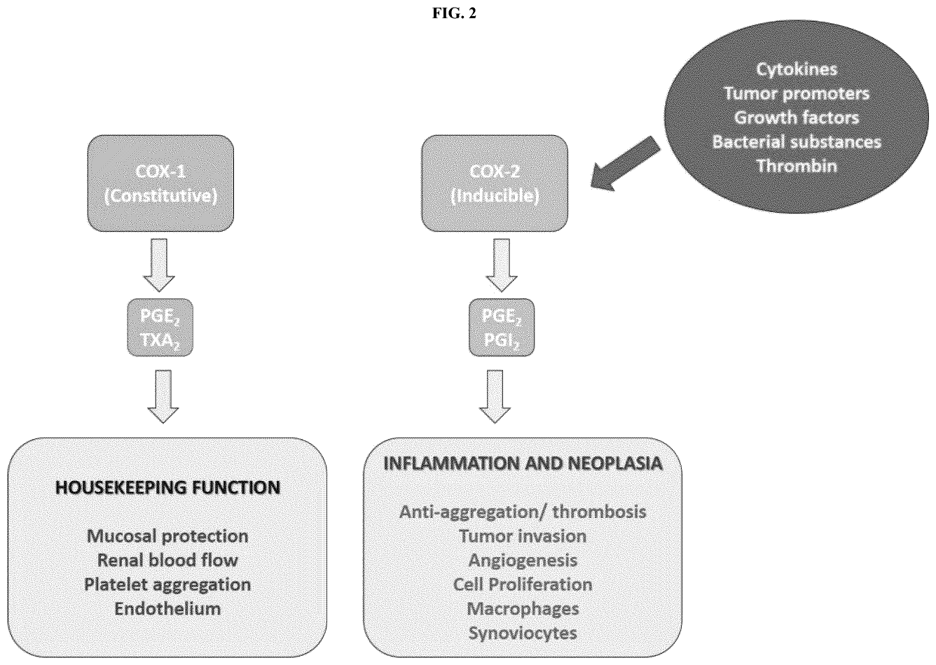

FIG. 3 presents an illustration of tryptophan metabolism in both non-stressful and stressful conditions. There is evidence that lipopolysaccharides (LPS) and pro-inflammatory cytokines increase tryptophan uptake in the brain and serotonin turnover.

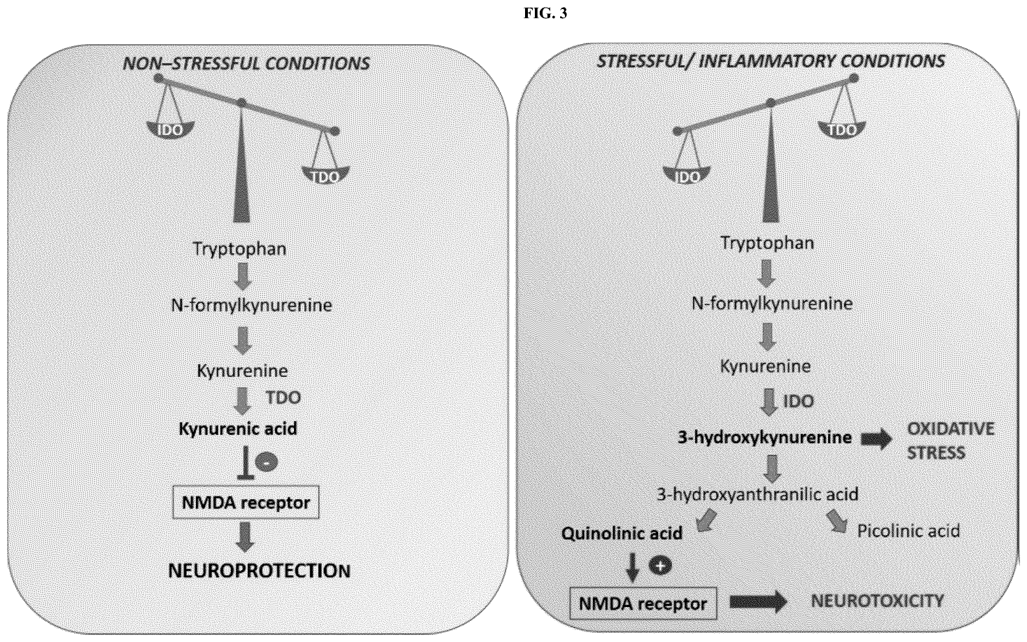

FIGS. 4A-4B show the toxicity results for Ziptamin toxicity tests. FIG. 4A is a male specimen and FIG. 4B is a female specimen. Neither show any toxic side effects from Ziptamin.

### DEFINITIONS

As used in the specification and claims, the singular form “a”, “an”, and “the” include plural references unless the context clearly dictates otherwise. For example, the term “a cell” includes a plurality of cells, including mixtures thereof.

As used in the specification and claims, the term “effective amount” regarding the disclosed composition means an amount sufficient to alleviate the symptoms associated with the condition being treated. The effective amount will depend upon the severity of the symptoms and on the responsiveness of the patient to the disclosed composition.

As used in the specification and claims, “treating”, “treatment” and the like refer to any and all applications which remedy, or otherwise hinder, retard, or reverse the progression of, a disorder or at least one symptom of a disease, including reducing the severity of a disorder. Thus, treatment does not necessarily imply that a subject is treated until complete elimination of, or recovery from, a disorder. Similarly, the terms “inhibiting”, “inhibition” and the like refer to any and all applications that inhibit the establishment of a disorder or otherwise delay or retard the onset of a disorder or a symptom thereof.

As used in the specification and claims, “co-administered” means simultaneous administration in the same composition or formulation or in two different compositions or formulations by the same or different routes. By “sequential” administration means a time difference of from seconds, minutes, hours or days between the administration of the agents, compositions or treatments. Sequential administration may be in any order. For combination treatment containing multiple compositions or formulations, those skilled in the art will appreciate that compositions formulation form (e.g., unit dosage forms) comprising the different agents or components to be administered need not be of the same type.

As used in the specification and claims, the term “neuroinflammation” is involved in the pathophysiology of depression by increasing proinflammatory cytokines, activating the hypothalamus-pituitary-adrenal axis, increasing glucocorticoid resistance, and affecting serotonin synthesis and metabolism, neuronal apoptosis and neurogenesis, and neuroplasticity.

As used in the specification and claims, the word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

As used in the specification and claims, “depression” refers to a clinical disorder characterized by a predominant sad or depressed mood and associated psychological and/or physical symptoms, which may present as a depressive or anxiety-related disorder. The disorder may manifest as mild, moderate, severe, clinical, or postpartum depression, and may include but is not limited to major depressive disorder, dysthymic disorder, seasonal affective disorder, mood disorder, mixed anxiety-depressive disorder, bipolar disorder, generalized anxiety disorder, and post-traumatic stress disorder. The methods and compositions described herein are applicable for the treatment of these and other similar conditions.

As used in the specification and claims, the term “treating and inhibiting depression, anxiety, or a depressive or anxiety-related disorder” refers to the reduction, partial or complete, of one or more symptoms associated with these conditions. Such symptoms may include, but are not limited to, irritability, mood changes, low mood, sleep disturbance, lethargy, short-term memory loss, anxiety, restlessness, tension, low self-esteem, and suicidal thoughts or tendencies. Additionally, “treating or inhibiting” encompasses avoiding the development of depression, anxiety, or a depressive or anxiety-related disorder in individuals who may be prone to such conditions or show symptoms but have not yet been diagnosed. It also includes inhibiting the onset of depressive or anxiety episodes in affected individuals.

“Treatment-resistant depression” refers to any form of depression or a related disorder that does not respond to at least one adequate trial of antidepressant therapy or responds less effectively than depression or a related disorder in a subject without resistance to the treatment. This can manifest as complete or partial resistance to treatment. An individual with depression or a related disorder that does not respond to a specific treatment may be referred to as a “non-responder,” while an individual with depression or a related disorder that responds to a lesser extent than those without resistance may be referred to as a “suboptimal responder” (displaying a “suboptimal treatment response”).

Though depressive feelings are common, depression or a depressive disorder is typically diagnosed only when the symptoms reach a threshold and last at least two weeks. There exist a number of methods and techniques well known to those skilled in the art for diagnosing depression, anxiety and depressive or anxiety-related disorders, for assessing the status or severity of such conditions or symptoms thereof over time, and for monitoring the change in status or severity of such conditions or symptoms thereof over time, including in response to treatment or therapy.

Exemplary self-assessment or self-report questionnaires include, but are not limited to the Depression and Anxiety Stress Scale (DASS), the Outcome Quiestionnaire-45 (OQ45), Quality of Life in Depression Scale (QLDS, including a Quality of Life (QoL) score), the Beck Depression Inventory (BDI), the Warwick-Edinburgh Mental Well-Being Scale (WBS), the Mini International Neuropsychiatric Interview (MINI), the Structured Clinical Interview for DSM Disorders (SCID) and the Patient Health Questionnaire (PHQ, such as PHQ-9 and PHQ-2). Exemplary clinician-completed reports or questionnaires include, but are not limited to, the Hamilton Depression Rating Scale (HAM-D) and the Raskin Depression rating Scale. Biochemical measurements that may be employed include, but are not limited to, whole blood serotonin levels.

OQ45 is an important measure used for treatment response tracking in both psychological and health research and practice and has proved a high valid measurement system. OQ45 is a self-report symptom and distress inventory designed as an independent measure of symptom distress and functioning to assess the response to intervention at regular intervals such as on a weekly basis (α=0.93, & κ>0.83). The OQ45 consists of 45 items on a five-point scale. A high total score <80 indicates a high level of symptom distress (anxiety, depression, and somatic, work and social role problems). Lower scores indicate less severity of problems. Average community non-clinical scores cut off (CO) occur at about >63, and changes of 14 points in either direction are typically considered clinically significant change and are reliable.

BDI is a 21-item self-report inventory that is widely used to assess depression. It has high internal constancy and correlates highly with other self-report measures of depression (α=0.60-0.90).

The DASS short form is a 21 item self-report scale designed to measure depression and anxiety and was used here as a secondary self-report assessment. The DASS has high internal consistency and yields meaningful discriminations in a variety of settings (α=0.88-0.96).

WBS has 14 items and assesses feelings and thoughts associated with mood (α=0.91) with higher scores indicate better mood. It has a different factor structure to the BDI and DASS and was used in this study as a measure of mood improvement.

QoL has 16 items and evaluates perceived satisfaction with life over a number of domains (α=0.85). QOL typically provides an important evaluation of perception of life circumstances and stressors.

The SCID clinical interview has acceptable validity in diagnosing personality disorder κ>0.75.

Eastern medicine often employs several herbs in its formulas. One popular method for arranging these herbs is based on a monarchy-style hierarchy, with the “king” herb serving as the main ingredient for addressing the syndrome being treated, the “minister” or “deputy” herb supporting the king herb by reinforcing its action and treating any concurrent symptoms, the “assistant” herb aiding the king herb, controlling its side effects, or reducing its toxicity, and the “servant” herb directing the formula to the affected area and harmonizing the other ingredients in the formula. See, published U.S. Pat. No. 2004/0180104 A1.

The above-mentioned Eastern medicine theory can be found at the website: www.tcm.health-info.org, www.chinesemedicineSampler.com, www.acuhealing.com, http://website.lineone.net/-balloonz/enhc.htm etc.

### DETAILED DESCRIPTION

The composition disclosed herein may be used in the treatment of immune anti-inflammatory conditions including, but not limited to, respiratory antiinflammation, neurological diseases, and anti-aging. A major advantage of the disclosure is that it is rarely accompanied by side effects compared with many other prescribed medications. Despite the clearly devastating effects of depression and anxiety, it is thought that more than half of all sufferers either do not seek medical treatment or fail to take appropriate prescribed or recommended medication. In addition to the social stigma attached to depression and anxiety, a significant factor in this lack of compliance is the detrimental side effects of existing options for anti-depressant medication.

### Composition and Uses of the Composition

The present disclosure provides compositions and methods directed to for the inhibiting or treating or both of a neurological condition such as neuroinflammation, a neurological disease, or cognitive function decline.

In one aspect, the disclosure provides a method for the inhibiting or treating or both of a neurological condition, the method comprising administering to a subject in need thereof a composition comprising a disclosed composition.

In some embodiments, administered compositions may comprise eight or more of the following extract, powder, liquid, bee or juice products: ginseng (Panax quinquefolium); ginger (Zingiber officinale Roscoe); turmeric (Curcuma longa); lemongrass (Cymbopogon); at least one or more sage products of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii); at least one or more oregano products of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus); goatweed (Scoparia Dulcis); thyme (Thymus vulgaris); lavender (Lavendula); chamomile (Matricaria chamomilla); black pepper (Piper nigrum); Propolis; milk thistle (Silybum marianum); ginkgo (Ginkgo biloba); candyleaf (Stevia rebaudiana); monk fruit (Siraiti grosvenorii); corn silk; cogongrass (Imperata cylindrica); peppermint (Mentha piperita); honey; and liquorice (Glycyrrhiza glabra).

In some embodiments, administered compositions may comprise the following extract, powder, liquid, bee or juice products: ginger (Zingiber officinale Roscoe); turmeric (Curcuma longa); lemongrass (Cymbopogon); at least one or more sage product of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii); at least one or more oregano product of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus); ginseng (Panax quinquefolium); black pepper (Piper nigrum); and honey.

In one aspect, the composition may comprise one to six parts ginger, two to five parts ginger, three to four parts ginger, or 3.5 parts ginger. In another embodiment, the composition may comprise one to six parts turmeric, two to five parts turmeric, three to four parts turmeric, or 3.5 parts turmeric. In some embodiments, the composition may comprise one to six parts lemongrass, two to five parts lemongrass, three to four parts lemongrass, or 3.5 parts lemongrass. In some embodiments, the composition may comprise one to eight parts oregano product, two to seven parts oregano product, three to six parts oregano product, four to five parts oregano product, or 4.5 parts oregano product. In some embodiments, the composition may comprise one to eight parts sage product, two to seven parts sage product, three to six parts sage product, four to five parts sage product, or 4.5 parts sage product. In some embodiments, the composition may comprise one part ginseng, one to eight parts ginseng, two to seven parts ginseng, three to six parts ginseng, four to five parts ginseng, 4.5 parts ginseng, or eight parts ginseng. In some embodiments, the composition may comprise one part black pepper, one to eight parts black pepper, two to seven parts black pepper, three to six parts black pepper, four to five parts black pepper, 4.5 parts black pepper, or eight parts black pepper. In some embodiments, the composition may comprise one to five parts honey, two to four parts honey, or three parts honey.

In further aspects, the composition may comprise one to six parts ginger, one to six parts turmeric, one to six parts lemongrass, one to eight parts oregano, one to eight parts sage, one to eight or eight parts ginseng, one or one to eight parts black pepper, and one to five parts honey. In further aspects, the composition may comprise two to five parts ginger, two to five parts turmeric, two to five parts lemongrass, three to six parts oregano, three to six parts sage, three to six or eight parts ginseng, one or three to six parts black pepper, and two to three parts honey. In still further aspects, the composition may comprise three to four parts ginger, three to four parts turmeric, three to four parts lemongrass, four to five parts oregano, four to five parts sage, four to five or eight parts ginseng, one or four to five parts black pepper and two to three parts honey. In further aspects, the composition may comprise 3.3-3.7 parts ginger, 3.3-3.7 parts turmeric, 3.3-3.7 parts lemongrass, 4.3-4.7 parts oregano, 4.3-4.7 parts sage, 4.3-4.7 or eight parts ginseng, one or 4.3-4.7 parts black pepper, and 2.8 to 3.2 parts honey.

Embodiments of the present disclosure may be in the form of, but not limited to, tea, foods, dietary supplements, or medicine. In some embodiments, the subject may be human.

In some embodiments, the neurological condition may be neuroinflammation, a neurological disease, or cognitive function decline. In some embodiments, the neurological disease may be a degenerative nerve condition or a mental disorder. In some embodiments, the degenerative nerve condition may be associated with Alzheimer's disease, Hunting's disease, Parkinson's disease, or amyotrophic lateral sclerosis. In some embodiments, the mental disorder may be depression, anxiety, schizophrenia or posttraumatic stress disorder. In some embodiments, the cognitive function decline may be learning ability decline and administering the composition improves memory or focus or both.

In some embodiments, the administration of a disclosed composition results in stimulation of the immune system of the subject. In some embodiments, stimulation of the immune system results in reduced inflammation in the subject.

In some embodiments, administration of a disclosed composition may inhibit expression of 5-lipoxygenase (5-LO), cyclooxygenase (CO), pro-inflammation cytokines (interleukin (IL)-1β, IL-6, and tumor necrosis factor (TNF)α), chemokines (CCL2, CCL5, CXCL1), secondary inflammatory mediators such as nitric oxide (NO) and prostaglandins (PGs), and/or reactive oxygen species (ROS). In some embodiments, administration of a disclosed composition may inhibit or lessen the proliferation of bacteria, viruses, and fungi.

### Preparation and Uses of Herb Compositions

In another aspect, the disclosure provides a composition which may be a food composition in some embodiments. In other embodiments, the composition is a pharmaceutical composition. When compositions of the present disclosure are pharmaceutical compositions, the administration route is not specifically limited, but they preferably have a dosage form suitable for, but not limited to, oral, nasal, parenteral, or vaccine-based administration. Pharmaceutical compositions of the present disclosure can be in various forms. For example, dosage forms suitable for oral administration include, but are not limited to, tablets, capsules, soft gels, powders, granules, pills, liquids, sachet, emulsions, suspensions, solutions, spirits, syrups, extracts, elixirs, and decoction.

For the powder form, the components of the herbal composition of the present disclosure may be air-dried, freeze dried, and/or finely ground. Formulations may contain various pharmaceutically acceptable carriers including, but not limited to, excipients, binders, disintegrating agents, lubricants, flavors, colorants, sweeteners, corrigents, solubilizing agents, suspending agents, emulsifiers, additives, coating agents, vitamin, and antioxidants. The pharmaceutical composition of the present disclosure further includes, in addition to the above, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium Stearate, mineral oil, etc., but are not limited thereto.

In some embodiments, the pharmaceutical or nutritional composition may comprise at least eight of the following extract, powder, liquid, bee or juice products: ginseng (Panax quinquefolium); ginger (Zingiber officinale Roscoe); turmeric (Curcuma longa); lemongrass (Cymbopogon); at least one or more sage products of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii); at least one or more oregano products of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus); goatweed (Scoparia Dulcis); thyme (Thymus vulgaris); lavender (Lavendula); chamomile (Matricaria chamomilla); black pepper (Piper nigrum); Propolis; milk thistle (Silybum marianum); ginkgo (Ginkgo biloba); candyleaf (Stevia rebaudiana); monk fruit (Siraiti grosvenorii); corn silk; cogongrass (Imperata cylindrica); peppermint (Mentha piperita); honey; and liquorice (Glycyrrhiza glabra).

In some embodiments, the pharmaceutical or nutritional composition may comprise the following extract, powder, liquid, bee or juice products: ginger (Zingiber officinale Roscoe); turmeric (Curcuma longa); lemongrass (Cymbopogon); at least one or more sage product of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii); at least one or more oregano product of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus); ginseng (Panax quinquefolium); black pepper (Piper nigrum); and honey.

In one aspect, the pharmaceutical or nutritional composition may comprise one to six parts ginger, two to five parts ginger, three to four parts ginger, or 3.5 parts ginger. In another embodiment, the composition may comprise one to six parts turmeric, two to five parts turmeric, three to four parts turmeric, or 3.5 parts turmeric. In some embodiments, the composition may comprise one to six parts lemongrass, two to five parts lemongrass, three to four parts lemongrass, or 3.5 parts lemongrass. In some embodiments, the composition may comprise one to eight parts oregano product, two to seven parts oregano product, three to six parts oregano product, four to five parts oregano product, or 4.5 parts oregano product. In some embodiments, the composition may comprise one to eight parts sage product, two to seven parts sage product, three to six parts sage product, four to five parts sage product, or 4.5 parts sage product. In some embodiments, the composition may comprise one part ginseng, one to eight parts ginseng, two to seven parts ginseng, three to six parts ginseng, four to five parts ginseng, 4.5 parts ginseng, or eight parts ginseng. In some embodiments, the composition may comprise one part black pepper, one to eight parts black pepper, two to seven parts black pepper, three to six parts black pepper, four to five parts black pepper, 4.5 parts black pepper, or eight parts black pepper. In some embodiments, the composition may comprise one to five parts honey, two to four parts honey, or three parts honey.

In further aspects, the pharmaceutical or nutritional composition may comprise one to six parts ginger, one to six parts turmeric, one to six parts lemongrass, one to eight parts oregano, one to eight parts sage, one to eight or eight parts ginseng, one or one to eight parts black pepper, and one to five parts honey. In further aspects, the composition may comprise two to five parts ginger, two to five parts turmeric, two to five parts lemongrass, three to six parts oregano, three to six parts sage, three to six or eight parts ginseng, one or three to six parts black pepper, and two to three parts honey. In still further aspects, the composition may comprise three to four parts ginger, three to four parts turmeric, three to four parts lemongrass, four to five parts oregano, four to five parts sage, four to five or eight parts ginseng, one or four to five parts black pepper and two to three parts honey. In further aspects, the composition may comprise 3.3-3.7 parts ginger, 3.3-3.7 parts turmeric, 3.3-3.7 parts lemongrass, 4.3-4.7 parts oregano, 4.3-4.7 parts sage, 4.3-4.7 or eight parts ginseng, one or 4.3-4.7 parts black pepper, and 2.8 to 3.2 parts honey.

In some embodiments, the pharmaceutical or nutritional composition may further comprise one or more of the following: Saponin (Ginsenosides: proptopanaxadiol, protopanaxatriol, oleanane), Non-saponin: Saccharides (monosaccharides, disaccharides, trisaccharide, polysaccharides, fiber, pectin), Nitrogen-contaning compounds (protein, peptide, amino acid, nucleic acids, alkaloid), Fat-soluble components (liquid, fatty acid, polyacetylenes, phenolic compounds, essential oils, phytosterols, organic acid, terpenoid), Vitamins (water-soluble vitamins), minerals, Phenolic (gingerols, shogaols, and paradols, quercetin, zingerone, gingerenone-A, and 6-dehydrogingerdione), Terpene (beta-bisabolene, alpha-curcumene, zingiberene, alpha-farnesene, and beta-sesquiphellandrene), and Polyphenols (6-gingerol, 8-gingerol, and 10-gingerol; Curcuma longa (neral, isoneral, geranial, isogeranial, geraniol, geranyl acetate, citronellal, citronellol, germacrene-D, and elemol); Cymbopogon (Major components: Terpenes and Terpenoid, and vitamins A, C, E, folate, niacin, and riboflavin, protein, antioxidants, and mineral nutrients, and neral, isoneral, geranial, isogeranial, geraniol, geranyl acetate, citronellal, citronellol, germacrene-D, and elemol, alkane (Pentane 2,4-Dimethyl-anticancer), ester (Dodecanoic acid tert-butyl ester-antiashtmatics, antipruritics, antipsoriatic), phenol, carboxyaldehyde; Sage (phenolic acids (caffeic acid, vanillic acid, ferulic acid, rosmarinic acid), flavonoids (luteolin, apigenin, quercetin), monoterpenes (a-thujon, b-thujon, 1,8-cincole, camphor), diterpenes (carnosic acid, carnosol, manool, rosmadial), triterpenes (oleanoic acid, ursolic acid), sesquiterpenes (a-humulene, viridiflorol)); Scoparia Dulcis (nitrogen-containing compounds (2-Hydroxy-2H-1,4-benzoxazol-3-one, Benzoxazine, Benzoxazolinone, Coixol, 1-Hydroxy-6-methoxy-2-benzoxazolinon, 3,6-Dimethoxy-benzoxazolin-2(3H)-one, (2R)-2-(β-D-Glucopyranosyloxy)-7-methoxy-2H-1,4-benzoxazin-3(4H)-one, (2R)-2-(β-D-Glucopyranosyloxy)-4,7-dimethoxy-2H-1,4-benzoxazin-3(4H)-one, (2R)-7-Methoxy-2H-1,4 benzoxazin-3(4H)-one 2-O-β-galactopyranoside, 7-Methoxy-2,4-hydroxy-1,4-benzoxazin-3(2H)-one, Dextromoramide, 2-Heptadecyl-2-imidazoline, 1-Methyl-2-pyrrolidinone, N1-Acetylspermine, Cyclohexylamine, Procaine, Epinephrine, Norepinephrine), flavonoids (flavones: Scutellarin methylester, Scutellarin, 5,7,8,3′,4′,5′-Hexahydroxy-flavone glucuronide, 5-Hydroxy-6,7-dimethoxyflavone-4′-O-β-glucose, iso-Vitexin, 5,7-Dihydroxy-3′4′,6,8-tetrametoxyflavone, Acerosin, Nevadensin, 5,6,4′-Trihydroxyflavone 7-O-α-L-2,3-di-O-acetylrhamnopyranosyl-(1→6)-β-D-glucopyranoside, Apigenin 7-O-α-L-2,3-di-O-acetylrhamnopyranosyl-(1→6)-β-D-glucopyranoside, Apigenin 7-O-α-L-3-O-acetylrhamnopyranosyl-(1→6)-β-D-glucopyranoside, Acacetin, Apigenin, Cirsimarin, Cynaroside, Homoplantaginin, Linarin, Pectolinarin, Isorhoifolin, Vicenin-2, Vitexin, Luteolin, Scutellarein, Hispidulin, Apigenin-8-C-α-L-arabinopyranoside, Apigenin-7-O-glucuronide, Hispidulin-7-O-glucuronide, Cirsimaritin, Cirsiliol, Salvigenin, Scutellarein 7-O-α-glucuronamide.), flavonols (Morin, Dihydroxy-dimethoxyflavone, Hydroxy-tetramethoxyflavone, Dillenetin 3-O-(6″-O-p-coumaroyl)-β-D-glucopyranoside, Rutin, Quercetin, Kaempferol.), flavan-3-ols: catchin, isoflavones: naringin, diterpenoids (Scopadulcic acid A, 4-epi-Scopadulcic acid, Scopadulcic acid B, Scopadulciol, iso-Dulcinol, Scopadulcic acid C, Dulcidiol, Dulcinodal, Dulcinodiol, Scopadiol decanoate, 4-epi-7α-O-Acetylscoparic acid A, 7α-Hydroxyscopadiol, (7S)-4-epi-7-Hydroxyscoparic acid A, 7α-O-Acetyl-8,17β-epoxyscoparic acid A, neo-Dulcinol, Dulcinodal-13-one, 4-epi-7α-Hydroxydulcinodal-13-one, Scoparic acid A, Scoparic acid B, Scoparic acid C, Scoparic acid E, Scoparicol A, Scoparicol B, Scoparic acid D, Scopadulin, Scopanolal, Scopadiol, Phytol), triterpenoids (Friedelin, Glutinol, α-Amyrin, Dulcioic acid, Betulinic acid, Lupeol, Ifflaionic acid, Glutenol, Glutinone, Taraxerol), steroids (Daucosterol, Stigmasterol, β-Sitosterol), phenolics (Chlorogenic, Caffeic acid, Ferulic acid, Sinapic acid, p-Coumaric acid, Forsythoside G, Actcoside, Ferruginoside C, Gentisic acid), aliphatics (Mannitol, Hexalure, 2-Hexyldecanoic acid, 5Z-Eicosenoic acid, Methyl arachidate, Stearic acid, Methyl stearate); Thymus vulgaris (Phenolics acids (quercetin, ferulic acid, syringic acid, caffeic acid, rosmarinic acid, p-cumaric acid), Biphenyl compounds (4,4′-dihydroxy-5,5′-disopropyl-2, 2′-dimethylbiphenyl-3,6-dione, 5,5′-diisopropyl-2,2′-dimethylbiphenyl-3,4,30,4/-tetraone, 4′-hydroxy-5,5′-diisopropyl-2,2′-dimethylbiphenyl-3,4-dione), Flavonoids (Flavonols, Flavones, Flavonols, Flavone glycosides, Methyl flavones, Flavonols, Apigenin, Luteolin, Hesperitin, Rutin, Quercetin, Hesperidin, Kaempferol, Kaempferol-3-O-rutinoside), Essential oils (Limonene, Linalool, gamma-Terpinene, p-Cymene, Carvacrol, Thymol)); Lavendula (Phenols (Pyrogallol, Gallic acid, Protocatechuic acid, Catechol, Catechin, Chlorogenic acid, P-OH-Benzoic acid, Benzoic acid, Caffeic acid, Caffeine, Ferulic acid, Iso-Ferulic acid, Coumaric acid, 3,4,5-Methoxy cinnamic acid), Flavonoid (Rosmarinic, Hesperidin, Rutin, Quercetrin, Naringenin, Quercetin, Hesperetin, kaempferol, Apigenin), Essential oil (a-Pinene, Myrcenc, Limonene, 1,8 Cincol, Camphor, Linalool, Linalyl acetate, Terpine-4-ol, Terpincol, Betenol, Geraniol acetate, Nerol, Geraniol)); Matricaria chamomilla (α-bisabolol, bisabolol oxides A and B and chamazulene or azulenesse, farnesene and spiro-ether quiterpene lactones, glycosides, hydroxycoumarins, flavanoids (apigenin, lutcolin, patuletin, and quercetin), coumarins (herniarin and umbelliferone), terpenoids, and mucilage); Piper nigrum (fibers, starch, proteins, carbohydrates, lignans, alkaloids, flavonoids, phenols, amides, piperine, and essential oil (major compounds are sabinene, α-pinene and β-pinene, β-caryophyllene, phellandrene, limonene, linalool, citral), antioxidant (beta carotene, lauric, myristic and palmitic acids)); Propolis (Flavanoids (Flavone chrysin, flavonol galangin, and flavanonc pinocembrin), nonflavonoids (gallic acid, protocatechuic acid, and derivatives of cinnamic acid: caffeic, p-coumaric, and ferulic acids)); Silybum marianum (silybinin A and B, isosilybinin A and B, silychristin, and silydianin & flavonoid taxifolin, flavonoids (such as quercetin, dihydrokaempferol, kaempferol, apigenin, naringin, criodyctiol, chrysocriol), proteins, sugars (arabinose, rhamnose, xylose, glucose), tocopherol, sterols (cholesterol, campesterol, stigmasterol, sitosterol), and lipids in the form of triglycerides (linoleic, oleic and palmitic acid)); and Ginkgo biloba (flavonoids (quercetin, kaempferol, and isorhamnetin), terpenoids (bilobalide and ginkgolides), bioflavonoids (ginkgetin, sciadopitysin, and isoginkgetin), and organic acids (ginkgolic acid), Terpenoids, alkylphenols (ardols, cardanols, α-hydroxycardanols, urushiols, isourushiols, and alkylphenolic acids), carboxylic acid (ferulic acid, p-coumaric acid, protocatechuic acid, caffeic acid, p-hydroxybenzoic acid, m-hydroxybenzoic acid, vanillic acid, isovanillic acid, gallic acid, and sinapic acid), lignans, proanthycyanidins (prodelphinidin and procyanidin), polyprenols (chain of 14-24 isopentenyl units, and dilichols), polysaccharides (glucose, rhamnose, mannose, arabinose, and galactose)).

In another aspect, the disclosure provides a method of inhibiting or treating or both bacterial infection, viral infection, fungal infection, inflammation, chronic cough, neuroinflammation, depression, anxiety disorders, or stress, the method comprising administering to a subject in need thereof an effective amount of a disclosed composition. In some embodiments, the disclosure provides a method of inhibiting or treating or both nerve degeneration associated with aging, the method comprising administering to a subject in need thereof an effective amount of a disclosed composition. In some embodiments, the disclosed composition promotes blood circulation, reduces congealed blood, or both. In some embodiments, the subject is human.

For the extract, the components of the present disclosure can be extracted by solvents such as water or any other solvents which can extract active substances from the natural substances at a temperature about 22° C. to the boiling point or above, with pressure time range of 0-2000 bar and 20 minutes to 52 hours, respectively. The resulting extract can be optionally treated through filtration, concentration, and drying.

According to another embodiment of the disclosure, the powder composition is produced by drying the components and grinding the dried components and then mixing the resulting components to obtain the powder composition.

The herbal composition disclosed in this research can be formulated into unit dosages suitable for daily administration. The unit dosage comprises a therapeutically effective amount of each herbal extract for a single dose or multiple doses, depending on various factors such as age, size, and condition of the individual. The unit dosage can be administered on a daily basis over an extended period of time. The disclosure recommends administering a between 450 mg/day to a maximum of 25 g/day of the active herbal composition, with a portion of the dose preferably divided into two or more administration times throughout the day.

As per the method outlined in this disclosure, the various agents to be administered to a subject may be administered either sequentially or concurrently and may be included as part of the same or separate compositions or formulations. Additionally, these agents may be co-administered with one or more additional treatments or therapies aimed at treating immune inflammation and its related disorders, including neurological diseases, depression, anxiety, memory impairment, focus, nerve degeneration, aging, and other related conditions.

The present disclosure may further comprise vitamins and/or minerals and/or amino acids. The vitamins and minerals may be selected from, but not limited to: vitamins A, B, C, D, E, K, and calcium, chromium, copper, fluorine, iodine, iron, magnesium, manganese, molybdenum, phosphorus, potassium, selenium, sodium, and zinc. The amino acids may be selected from, but are not limited to: alanine, arginine, aspartic acid, cystine, glycine, histidine, lysine, methionine, phenylalanine, proline, serine, threonine, tryptophan and tyrosine.

The present disclosure can be combined with Ginkgo biloba extract, astragalus extract, guarana extract, dulcis extract, Apocynum venetum L. extract and/or melatonin to provide compositions in the form of tea, food, dietary supplement, medicine, or the like having the effect of improving depression caused by mental stress.

The present disclosure can be combined with extracts of black cohosh extracts, pumpkin Seed extracts, Soybean extracts, licorice extracts, Dong Quai extracts, chaste tree extracts, pomegranate extracts, and Dioscorea extracts to provide compositions in the form of a tea, food, dietary Supplement, medicine or the like having the effect of improving climacteric melancholia.

The present disclosure can be combined with extracts of ivy leaf, Andrographis paniculate, Pouzolzia Zeylanica for respiratory inflammation, chronic cough, COPD and asthma, and help to reduce side effect of Covid and flu vaccine.

The present disclosure can be combined with antibiotic drugs or antiviral drugs or antifungal drugs for chronic cough with color phlegm or related diseases for quicker recovery, reduce side effects and result in a better lifestyle.

The present disclosure can be combined with extracts of antihistamin for environmental change and weather change.

The present disclosure can be combined with vitamin B, L-tyrosine, L-Theamine, Phosphatidylserine, L-alpha glycerylphosphorylcholine, Bacoba, toothe clubmoss to treat dementia and related health condition.

The present disclosure can be combined with extracts of mushroom, seaweed, and/or probiotics for gut balance, anti-cancer, immune enhancement, reduce depression and psychiatric illnesses.

The present disclosure can be combined with extracted Tribulus Terrestris, Lepidium Meyenii, Horny goat, Maca root, Tribulus terrestris, Beetroot, Pine bark, Ginkgo biloba, L-arginine to treat erectile disfunction.

The composition of the present disclosure may be formulated for administration by any suitable route, such as oral, or nasal administration, or by inhalation, or by parenteral. In some embodiments, a disclosed composition may be administered subcutaneously, intramuscularly, intravenously, or intrathecally.

For these purposes a composition may be formulated by means known in the art into the form of for examples, tablets, capsules, granules, pills, sachet, soft gel, liquid, solutions, oil solutions, suspensions, emulsions, nasal sprays, powders, finely divided powders, aerosols for inhalation, sprits, syrups, extract elixirs, or raw herbs decoction.

The present disclosure is also directed to a method inhibiting or treating of one or more of the following: skin inflammation, acne, dark spots, and wrinkles through administration to a subject in need thereof an effective amount of a disclosed composition. In some embodiments, the administration of a disclosed composition may be topical. In some embodiments, the administration of a disclosed composition may be oral. In some embodiments, the subject may be human.

### EXAMPLES

### Example 1: Oral Acute Toxicity Survey on Mice

Procedure: Administer to 10 mice (5 males, 5 females) who have fasted for at least 12 hours before oral administration of the maximum possible test dose, with a volume of 50 ml/kg per mouse according to the Guidelines for preclinical and clinical testing of traditional medicine, herbal medicine by the Ministry of Health (2015) and by Do Trung Dam (2014), “Method for determining the acute toxicity of drugs”, Medical Publishing House, Hanoi. Monitor and record general movements, behavioral expressions, fur condition, eating, excreting, and the number of deaths within 72 hours. If after 72 hours, mice show no abnormal signs or death, continue observation for 14 days. Three possible outcomes:
Case 1: If all test mice survive after administration, determine the highest possible dose of the test sample that does not cause death (Dₘₐₓ).
Case 2: If 100% of the mice die after administration, test with half the initial dose. Continue reducing the dose until finding the minimum lethal dose for 100% of the mice (LD₁₀₀) and the maximum non-lethal dose (LD₀). Proceed to determine LD₅₀.
Case 3: If less than 100% of the mice die after administration, LD₁₀₀ cannot be determined, nor can LD₅₀. In this case, the maximum non-lethal dose can be determined, known as LD₀.

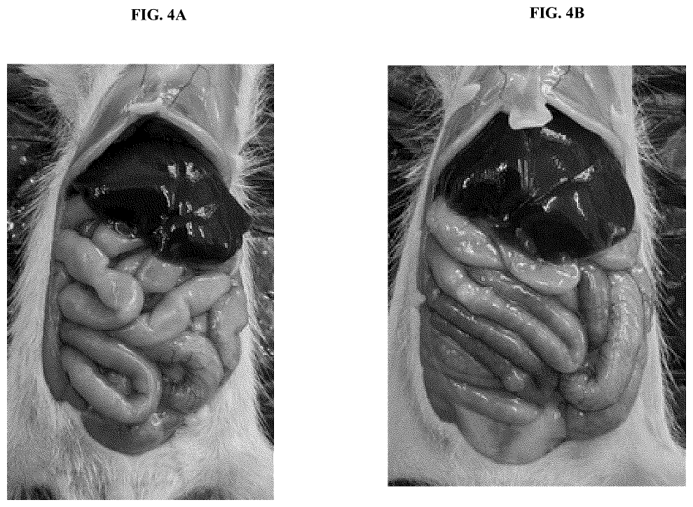

Results: The oral acute toxicity of Ziptamin was surveyed on 10 white mice (5 males, 5 females). Mice were given Ziptamin 4 times/day with a maximum possible volume of 50 ml/kg, 3 hours apart between doses. Mice showed no signs of toxicity after Ziptamin administration, moved normally, were healthy, ate feed, drank water, excreted, and had normal movements with no deaths within the first 72 observation hours. During the 14-day observation period, no deaths were observed; all surviving mice showed no abnormal behavioral signs, fur condition, eating, or excreting. Gross anatomical results shown in FIGS. 4A and 4B revealed no abnormalities in the heart, lungs, liver, kidneys, or digestive system of mice after the 14-day trial. Thus, Ziptamin does not exhibit acute oral toxicity in mice with the maximum feasible oral dose of 200 ml/kg/day, corresponding to 1020 ml/day in a 60 kg human. Results are summarized in Table 1.

TABLE 1
Results of the Oral Acute Toxicity Test in Mice of Ziptamin Product

| Experimental group | 1 | 2 | 3 | 4 | 5 | 6 | 7 | 8 | 9 | 10 |
|---|---|---|---|---|---|---|---|---|---|---|
| Gender | ♂ | ♂ | ♂ | ♂ | ♂ | ♀ | ♀ | ♀ | ♀ | ♀ |
| Weight (g) | 21.0 | 20.7 | 20.6 | 18.7 | 18.6 | 22.7 | 20.9 | 21.1 | 19.7 | 20.0 |
| Volume per | 1.05 | 1.04 | 1.03 | 0.94 | 0.93 | 1.14 | 1.05 | 1.06 | 0.99 | 1.00 |
| administration (ml) | | | | | | | | | | |
| Volume for 4 | 4.20 | 4.14 | 4.12 | 3.74 | 3.72 | 4.54 | 4.18 | 4.22 | 3.94 | 4.00 |
| administrations (ml) | | | | | | | | | | |
| Total weight (g) | | | 204 | | | | | | | |
| Total volume (ml) | | | 40.8 | | | | | | | |
| Number of test mice | | | 10 | | | | | | | |
| Number of mice | | | 0 | | | | | | | |
| deceased after 72 hrs | | | | | | | | | | |
| Number of mice | | | 0 | | | | | | | |
| deceased after 14 days | | | | | | | | | | |
| Weight after 72 hrs (g) | 22.6 | 22.3 | 23.4 | 20.0 | 19.6 | 23.1 | 24.5 | 22.2 | 20.8 | 21.2 |
| Weight after 7 days (g) | 26.2 | 26.4 | 25.5 | 22.3 | 21.9 | 23.5 | 25.0 | 23.0 | 22.3 | 22.0 |
| Weight after 14 days (g) | 31.6 | 30.4 | 30.7 | 29.3 | 27.3 | 29.5 | 27.7 | 26.7 | 27.6 | 23.7 |

### Example 2: Survey on Immune Stimulatory Effects on Mice with Immunosuppression Induced by Cyclophosphamide [3, 4]

### Test Doses:

The adult human dosage of Ziptamin is 0.5-1 bottle/day×250 ml/bottle A normal adult weighs about 60 kg→Mouse dose of Ziptamin: Dₘₒᵤₛₑ=(0.5*250*11.76)/60=25 ml/kg and Dmouse=(1*250*11.76)/60=50 ml/kg. The dose conversion factor from adults to mice is 11.76 according to the Guidelines for preclinical and clinical testing of traditional medicine, herbal medicine issued by the Department of Science, Technology and Training-Ministry of Health under Decision No. 141/QD-K2DT dated 27 Oct. 2015.

### Procedure:

Healthy male mice, after stable housing, were randomly divided into 5 groups, each with 8 mice:
Group 1 (Physiological): Mice were given distilled water;
Group 2 (Disease control): Mice were given distilled water;
Group 3 (Control): Mice were injected intraperitoneally with filgrastim at a dose of 50 μg/kg;
Group 4 (Test group): Mice were given Ziptamin at a dose of 25 ml/kg, divided into 2 doses/day 4 hours apart;
Group 5 (Test group): Mice were given Ziptamin at a dose of 50 ml/kg, divided into 2 doses/day 4 hours apart;
Group 1 (physiological) were injected intraperitoneally with 0.9% NaCl, volume 10 ml/kg. Mice from groups 2 to 5 were injected intraperitoneally with CYP (dissolved in 0.9% NaCl) at a single dose of 150 mg/kg on day 1, injection volume 10 ml/kg. Mice in group 3 were subcutaneously injected with filgrastim (dissolved in 5% glucose), dose 50 μg/kg, once a day, from day 2 to day 4, the first dose 24 hours after CYP injection. Mice in groups 4 and 5 were given Ziptamin at doses of 25 ml/kg and 50 ml/kg from day 1 to day 4, divided into 2 doses/day, 4 hours apart between doses. Mice in the test groups were recorded for weight daily before administering distilled water, test sample, or medication. On day 4, one hour after the filgrastim injection or Ziptamin administration, mice were euthanized with CO2 gas, blood was collected from the heart to determine the leukocyte formula at Medlatec clinic; spleen and thymus were collected, rinsed with cold 0.9% NaCl, dried, weighed, and the relative organ mass was determined using the formula:

$$g \% = {P}_{o r g a n} / {P}_{body} * 100$$

Where:
P_{organ}: organ weight (g)
P_{body}: body weight (g)

### Result Processing and Statistical Analysis

Results are presented as mean±SEM (standard error of mean). Differences between groups were analyzed using Kruskal-Wallis and Mann-Whitney tests with SPSS 26.0 software. A difference was considered statistically significant when the p-value <0.05.

### Results: Effect on the Body Weight of Test Mice:

The average body weight of test mice in each group is presented in Table 2. The average weight of mice in the physiological group increased by 0.5-1 g/day, while the disease control group varied unevenly around 0.1-1.8 g/day, with no significant difference compared to the physiological group at the same time point (p>0.05).

Mice treated with filgrastim 50 μg/kg as well as Ziptamin doses of 25 ml/kg and 50 ml/kg had a body weight decrease of 0.5-2 g/day, lower than the disease control and physiological groups, but not statistically significant (p>0.05). Therefore, administering Ziptamin to mice at doses of 25 ml/kg and 50 ml/kg divided into two daily administrations, with a 4-hour interval between doses, did not demonstrate an effect on improving the body weight of mice with immunosuppression induced by cyclophosphamide.

TABLE 2
Body weight (g) of mice in each test group

| Group (n = 8) | Day 1 | Day 2 | Day 3 | Day 4 |
|---|---|---|---|---|
| Normal | 19.46 ± 1.01 | 21.73 ± 1.97 | 19.75 ± 0.91 | 20.18 ± 0.96 |
| Disease control | 20.13 ± 1.74 | 20.24 ± 2.47 | 18.48 ± 2.48* | 19.03 ± 2.47 |
| Filgrastim 50 μg/kg | 19.34 ± 0.97 | 18.19 ± 1.21** | 17.64 ± 1.41** | 17.08 ± 1.52** |
| Ziptamin 25 ml/kg | 20.56 ± 1.23 | 19.58 ± 1.51* | 18.48 ± 1.10* | 17.05 ± 1.54** |
| Ziptamin 50 ml/kg | 20.01 ± 1.76 | 17.94 ± 2.22** | 19.18 ± 1.01^{$} | 18.46 ± 1.12* |

*p < 0.05 and
**p < 0.01 compared to the physiological group at the same time of the study
^{$}p < 0.05 compared to the filgrastim group at the same time of the study

### Results: Impact on the Relative Weight of the Spleen and Thymus:

The thymus and spleen are specialized organs of the immune system. The results on the relative weight of the spleen and thymus in the test groups are presented in Table 3.

TABLE 3
Relative weight of the spleen and thymus
(% g/g) mice of the test groups

| Experimental group | Relative weight of the | Relative weight of the |
|---|---|---|
| (n = 8) | spleen | thymus |
| Normal | 0.47 ± 0.03 | 0.15 ± 0.03 |
| Disease control | 0.14 ± 0.03** | 0.07 ± 0.02** |
| Filgrastim 50 μg/kg | 0.16 ± 0.05** | 0.06 ± 0.03** |
| Ziptamin 25 ml/kg | 0.15 ± 0.05** | 0.06 ± 0.01**^{#} |
| Ziptamin 50 ml/kg | 0.14 ± 0.04** | 0.05 ± 0.02** |

**p < 0.01 compared to the physiological group at the same time of the study
^{#}p < 0.05 compared to the disease control group at the same time of the study

Mice with immunosuppression induced by cyclophosphamide injection exhibited lower relative weights of the spleen and thymus, respectively 3.37 times and 2.12 times lower than that of physiological mice at the same time of the study (p<0.01). This indicates that intraperitoneal injection of cyclophosphamide at a dose of 150 mg/kg on day 1 caused damage to the immune organs of the test mice.

At the end of the experiment, compared to the disease control group, the group of mice treated with positive control filgrastim at a dose of 50 μg/kg and Ziptamin at a dose of 25 ml/kg showed an increase in the relative weight of the spleen by 0.01-0.02, and the weight of the thymus remained unchanged; the difference was not significant (p>0.05).

### Results: Impact on the Number and Formula of White Blood Cells

TABLE 4
The number and formula of white blood cells (WBC) in mice of the test groups

| Group | | Volume WBC (10⁹/L) | | | Ratio WBCC (%) | | |
|---|---|---|---|---|---|---|---|
| (n = 8) | Total WBC | Neutral | Lympho | Mono | Neutral | Lympho | Mono |
| Normal | 7.44 ± 1.16 | 0.88 ± 0.17 | 5.11 ± 0.93 | 1.45 ± 0.23 | 10.45 ± 1.34 | 69.29 ± 3.25 | 20.26 ± 2.76 |
| Disease control | 1.75 ± 0.39** | 0.15 ± 0.05** | 1.56 ± 0.35** | 0.04 ± 0.05** | 2.49 ± 0.90** | 92.13 ± 2.64** | 5.39 ± 2.13** |
| Filgrastim | 2.66 ± 0.73**^{#} | 0.25 ± 0.13** | 2.26 ± 0.60**^{#} | 0.15 ± 0.10**^{#} | 5.74 ± 2.84*^{##} | 87.26 ± 4.57**^{#} | 7.00 ± 1.91** |
| 50 μg/kg | | | | | | | |
| Ziptamin 25 | 1.69 ± 0.50**^{$$} | 0.26 ± 0.15** | 1.33 ± 0.37**^{$$} | 0.10 ± 0.05** | 8.45 ± 4.70^{##} | 81.53 ± 6.80**^{##} | 10.03 ± 3.13**^{##$} |
| ml/kg | | | | | | | |
| Ziptamin 50 | 2.13 ± 0.52** | 0.24 ± 0.11** | 1.71 ± 0.36**^{$} | 0.18 ± 0.12**^{#} | 6.74 ± 4.27*^{##} | 83.34 ± 7.66**^{##} | 9.93 ± 3.68**^{#} |
| ml/kg | | | | | | | |

*p < 0.05 and **p < 0.01 compared to the physiological group at the same time of the study
^{#}p < 0.05 and ^{##}p < 0.01 compared to the disease control group at the same time of the study
^{$}p < 0.05 and ^{$$}p < 0.01 compared to the filgrastim group at the same time of the study

Compared to the physiological group, the cyclophosphamide-induced disease group showed a statistically significant lower number and formula of white blood cells (p<0.01), specifically: the total white blood cell count decreased by 4.3 times, the number and proportion of neutrophils decreased by 5.8 and 4.2 times; the number of lymphocytes decreased by 3.3 times but the proportion increased by 1.3 times; the number and proportion of monocytes decreased by 38.7 and 3.8 times. This indicates that intraperitoneal injection of cyclophosphamide at a dose of 150 mg/kg caused immunosuppression in mice.

Mice treated subcutaneously with the positive control drug filgrastim at a dose of 50 μg/kg had an increase in the total white blood cell count, lymphocyte count, and monocyte count, as well as the proportion of neutrophils and lymphocytes compared to mice in the disease control group (p<0.05).

Mice given Ziptamin at doses of 25 ml/kg and 50 ml/kg had an increase in the proportion of neutrophils, lymphocytes, and monocytes by 1.84-4.67 times compared to mice in the disease control group (p<0.05). The immunostimulatory effect of Ziptamin on improving the number and formula of white blood cells was similar to that of positive control filgrastim at 50 μg/kg (p>0.05) but did not normalize these parameters to the levels of the physiological group (p<0.01).

Therefore, the preliminary results show that Ziptamin given to mice at doses of 25 ml/kg and 50 ml/kg, divided into 2 doses per day, 4 hours apart (corresponding to a human dosage of 0.5-1 bottle/day×250 ml/bottle for an adult), for 5 consecutive days showed immunostimulatory effects, helping to increase the number and improve the formula of white blood cells in mice with immunosuppression induced by a single intraperitoneal injection of cyclophosphamide at a dose of 150 mg/kg.

Conclusion of these Tests:

Ziptamin did not show acute oral toxicity in mice at the maximum possible dose of 200 ml/kg/day, corresponding to 1020 ml/day in a 60 kg person. Ziptamin given to mice at doses of 25 ml/kg and 50 ml/kg, divided into 2 doses per day, 4 hours apart (corresponding to a human dosage of 0.5-1 bottle/day×250 ml/bottle for an adult), for 5 consecutive days showed immunostimulatory effects, helping to increase the number and improve the formula of white blood cells in mice with immunosuppression induced by a single intraperitoneal injection of cyclophosphamide at a dose of 150 mg/kg.

### Example 3: Forced Swimming Test (FST)

### Method: Investigating Anti-Anxiety and Stress Effects Through the Forced Swim Test

The anti-anxiety and stress effects of the Ziptamin product were investigated in mice using the forced swim test as reported in Porsolt, et al. (5). Healthy female mice, after acclimatization, were randomly divided into 4 groups, each group consisting of 6 mice, specifically:
Group 1 (Disease control): Mice were administered distilled water;
Group 2 (Control): Mice were administered fluoxetine at a dose of 6.5 mg/kg;
Group 3 (Test sample): Mice were administered Ziptamin at a dose of 25 ml/kg, divided into 2 doses/day 4 hours apart;
Group 4 (Test sample): Mice were administered Ziptamin at a dose of 50 ml/kg, divided into 2 doses/day 4 hours apart.

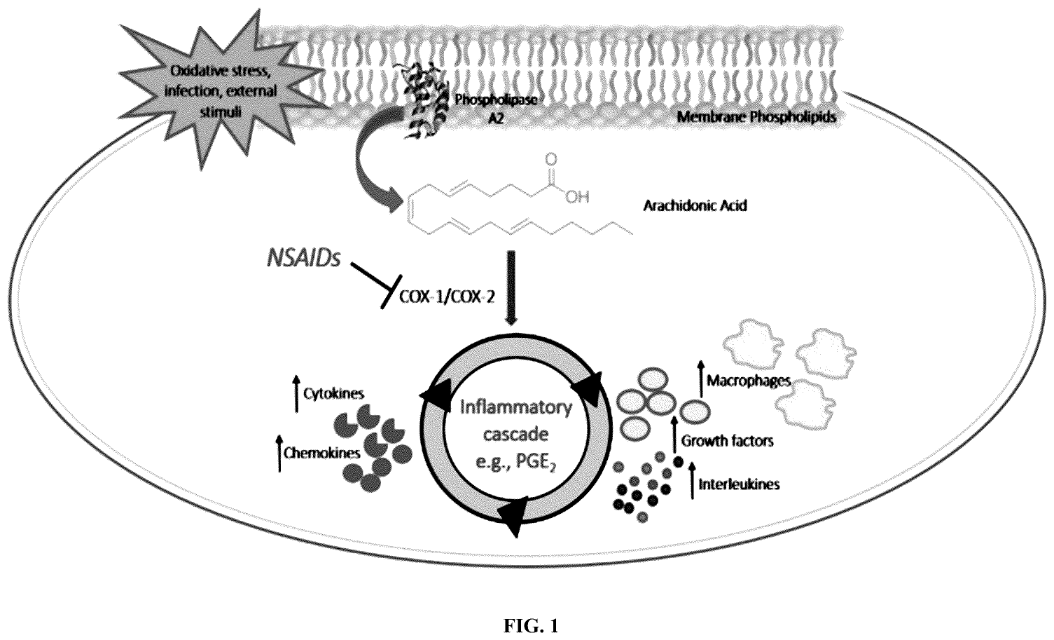

Mice were administered distilled water or fluoxetine at a volume of 10 ml/kg once a day or Ziptamin at doses of 25 ml/kg and 50 ml/kg for 7 consecutive days, divided into 2 doses/day, with a 4-hour interval between doses. On day 7, 60 minutes after administering distilled water, fluoxetine, or Ziptamin, mice were placed in a cylindrical glass tube (diameter 20 cm, height 50 cm) containing water at a temperature of 23-25° C. and a depth of 20 cm for 6 minutes. Initially, the mice struggled to escape the water, but then gradually adapted and performed only the necessary movements to keep their heads above water (immobility state). Filming lasted for 6 minutes, including 1 minute for the mice to stabilize and adapt, and the remaining 5 minutes to record the movements and behavior of the mice in the water, including swimming time (mice moving their limbs in the water as if rowing in the center of the cylinder), struggling (front limbs moving quickly and strongly in or above the water, with a tendency to swim and hit the front limbs against the walls of the cylinder), and immobility (ceasing to struggle, only performing necessary movements to keep the head afloat) (FIG. 1). The test sample reduced anxiety and stress, decreasing immobility time and increasing swimming and struggling time in the test mice. After 6 minutes, the mice were removed from the water and dried to prevent hypothermia. Results are presented as mean values±SEM (standard error of the mean). Differences between groups were analyzed using the Kruskal-Wallis test and Mann-Whitney test with SPSS 26.0 software. A statistical significance is considered when the p-value is <0.05.

### Results: Anti-Anxiety and Stress-Reducing Effects on Mice Through the Forced Swimming Test by Ziptamin Product

Swimming, struggling, and immobility times of mice in the experimental groups are presented in Table 5.

Mice exhibited anxiety and stress when placed in a water-filled environment, demonstrated by a higher immobility time, 1.39 times and 19.58 times higher respectively compared to swimming time and paddling time.

Mice administered a 6.5 mg/kg dose of fluoxetine showed a 90.27% increase in swimming time and a statistically significant 68.66% decrease in immobility time in water compared to the disease control group (p<0.01). The paddling time of the fluoxetine 6.5 mg/kg group was 1.77 times higher than the disease control group but not statistically significant (p>0.05). The results indicate that the positive control fluoxetine at a 6.5 mg/kg dose shows a response in mice with anxiety and stress using the forced swim test, thereby applying this model to investigate the anxiolytic and stress-reducing effects of Ziptamin.

TABLE 5
Swimming, struggling, and immobility times of
mice (seconds) in the experimental groups

| Experimental | | | |
|---|---|---|---|
| group | Time (second) | | |
| (n = 6) | Swimming | Struggling | Immobility |
| Control | 121.67 ± 65.50 | 8.67 ± 5.03 | 169.67 ± 63.82 |
| group | | | |
| Fluoxetin | 231.50 ± 28.25** | 15.33 ± 8.52 | 53.17 ± 29.27** |
| 6.5 mg/kg | | | |
| Ziptamin | 270.67 ± 8.52** | 14.00 ± 7.19 | 15.17 ± 6.99**^{$} |
| 25 ml/kg | | | |
| Ziptamin | 282.83 ± 9.21**^{$$} | 17.17 ± 9.21 | 0.00 ± 0.00**^{$$@@} |
| 50 ml/kg | | | |

**p < 0.01 compared to disease control
^{$}p < 0.05 and
^{$$}p < 0.01 compared to 6.5 mg/kg dose of fluoxetine
^{@@}p < 0.01 compared to 25 ml/kg dose of Ziptamin

The Ziptamin 25 ml/kg group showed a statistically significant increase in swimming time by 122.47% (p<0.01) and a decrease in immobility time by 91.06% (p<0.01) compared to the disease control group. In the group administered Ziptamin at a 50 ml/kg dose, mice continuously swam with no immobility time (p<0.01), and the swimming time increased by 132.47% (p<0.01) compared to the disease control group. The paddling time in water for both Ziptamin test doses increased by 61.54-98.08% compared to stressed mice, however, this difference was not statistically significant (p>0.05).

The results indicate that mice administered Ziptamin at doses of 25 ml/kg and 50 ml/kg showed a significant increase in swimming time and a decrease in immobility time compared to mice administered a 6.5 mg/kg dose of fluoxetine (p<0.05 and p<0.01). Comparing the two Ziptamin doses of 25 ml/kg and 50 ml/kg, there was no difference in swimming and paddling time (p>0.05), however, the immobility time in water for the Ziptamin 50 ml/kg dose was significantly lower than the Ziptamin 25 ml/kg dose (p<0.01).

Therefore, administering Ziptamin at doses of 25 ml/kg and 50 ml/kg to mice, divided into two daily doses 4 hours apart for 7 consecutive days, demonstrates an anxiolytic and stress-reducing effect on mice using the forced swim model, increasing swimming time and reducing immobility time in water; the effect of the 50 ml/kg dose of Ziptamin is better than that of the 25 ml/kg dose and the positive control fluoxetine at a 6.5 mg/kg dose.

### Conclusion

Administering Ziptamin at doses of 25 ml/kg and 50 ml/kg to mice, divided into two daily doses 4 hours apart for 7 consecutive days, demonstrates an anxiolytic and stress-reducing effect on mice using the forced swim model, increasing swimming time and reducing immobility time in water; the effect of the 50 ml/kg dose of Ziptamin is better than that of the 25 ml/kg dose and the positive control fluoxetine at a 6.5 mg/kg dose.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

(1) Müller N. COX-2 Inhibitors, Aspirin, and Other Potential Anti-Inflammatory Treatments for Psychiatric Disorders. Front Psychiatry. 2019 May 31; 10:375. doi: 10.3389/fpsyt.2019.00375. PMID: 31214060; PMCID: PMC6555131.
(2) Perrone M G, Centonze A, Miciaccia M, Ferorelli S, Scilimati A. Cyclooxygenase Inhibition Safety and Efficacy in Inflammation-Based Psychiatric Disorders. Molecules. 2020 Nov. 18; 25(22):5388. doi: 10.3390/molecules25225388. PMID: 33217958; PMCID: PMC7698629.
(3) Limbana T, Khan F, Eskander N. Gut Microbiome and Depression: How Microbes Affect the Way We Think. Cureus. 2020 Aug. 23; 12(8): e9966. doi: 10.7759/cureus.9966. PMID: 32983670; PMCID: PMC7510518.
(4) Shen, C.-Y., Jiang, J.-G., Yang, L., Wang, D.-W., and Zhu, W. (2017) Anti-ageing active ingredients from herbs and nutraceuticals used in traditional Chinese medicine: pharmacological mechanisms and implications for drug discovery. British Journal of Pharmacology, 174: 1395-1425. doi: 10.1111/bph.13631.
(5) Porsolt R D, Anton G, Blavet N, Jalfre M. Behavioural despair in rats: a new model sensitive to antidepressant treatments. Eur J Pharmacol. 1978 Feb. 15; 47(4):379-91. doi: 10.1016/0014-2999(78)90118-8. PMID: 204499.

What is claimed is:

1. A method for the inhibiting and/or treating neurological condition, the method comprising administering to a subject in need thereof a composition comprising:
at least eight extract, powder, liquid, bee or juice products selected from the group consisting of:
(i) ginseng (Panax quinquefolium);
(ii) ginger (Zingiber officinale Roscoe);
(iii) turmeric (Curcuma longa);
(iv) lemongrass (Cymbopogon);
(v) at least one or more sage products selected from the group consisting of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii);
(vi) at least one or more oregano products selected from the group consisting of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus);
(vii) goatweed (Scoparia Dulcis);
(viii) thyme (Thymus vulgaris);
(ix) lavender (Lavendula);
(x) chamomile (Matricaria chamomilla);
(xi) black pepper (Piper nigrum);
(xii) Propolis;
(xiii) milk thistle (Silybum marianum);
(xiv) ginkgo (Ginkgo biloba);
(xv) candyleaf (Stevia rebaudiana);
(xvi) monk fruit (Siraiti grosvenorii);
(xvii) corn silk;
(xviii) cogongrass (Imperata cylindrica);
(xix) peppermint (Mentha piperita);
(xx) honey; and
(xxi) liquorice (Glycyrrhiza glabra).

2. The method of claim 1, wherein the at least eight extract, powder, liquid, bee or juice products comprise:
(i) ginger (Zingiber officinale Roscoe);
(ii) turmeric (Curcuma longa);
(iii) lemongrass (Cymbopogon);
(iv) at least one or more sage products of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii);
(v) at least one or more oregano products of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus);
(vi) ginseng (Panax quinquefolium);
(vii) black pepper (Piper nigrum; and
(viii) honey.

3. The method of claim 2, wherein the composition is a pharmaceutical composition or food composition.

4. The method of claim 3, wherein the neurological condition is neuroinflammation, a neurological disease, cognitive function decline, or a combination thereof.

5. The method of claim 4, wherein the neurological disease is a degenerative nerve condition and/or a mental disorder.

6. The method of claim 5, wherein the degenerative nerve condition is associated with dementia, Alzheimer's disease, Hunting's disease, Parkinson's disease, or amyotrophic lateral sclerosis, or a combination thereof.

7. The method of claim 5, wherein the mental disorder is depression, anxiety, schizophrenia, posttraumatic stress disorder, or a combination thereof.

8. The method of claim 4, wherein the cognitive function decline is learning ability decline and administering the composition to the person improves memory, or focus, or both.

9. The method of claim 2, wherein the administration of the composition results in stimulation of the immune system of the subject.

10. The method of claim 9, wherein the stimulation of the immune system of the subject results in reduced inflammation.

11. A pharmaceutical or nutritional composition comprising:
at least eight extract, powder, liquid, bee or juice products selected from the group consisting of:
(i) ginseng (Panax quinquefolium);
(ii) ginger (Zingiber officinale Roscoe);
(iii) turmeric (Curcuma longa);
(iv) lemongrass (Cymbopogon);
(v) at least one or more sage products selected from the group consisting of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii);
(vi) at least one or more oregano products selected from the group consisting of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus);
(vii) goatweed (Scoparia Dulcis);
(viii) thyme (Thymus vulgaris);
(ix) lavender (Lavendula);
(x) chamomile (Matricaria chamomilla);
(xi) black pepper (Piper nigrum);
(xii) Propolis;
(xiii) milk thistle (Silybum marianum);
(xiv) ginkgo (Ginkgo biloba);
(xv) candyleaf (Stevia rebaudiana);
(xvi) monk fruit (Siraiti grosvenorii);
(xvii) corn silk;
(xviii) cogongrass (Imperata cylindrica);
(xix) peppermint (Mentha piperita);
(xx) honey; and
(xxi) liquorice (Glycyrrhiza glabra).

12. The pharmaceutical or nutritional composition of claim 11, wherein the at least eight extract, powder, liquid, bee or juice products comprise:
(i) ginger (Zingiber officinale Roscoe);
(ii) turmeric (Curcuma longa);
(iii) lemongrass (Cymbopogon);
(iv) at least one or more sage products of: Jerusalem sage (Phlomis fruticosa), common sage (Salvia officinalis), Greek sage (Salvia fruticosa), Grage sage (Salvia melissordora), grapefruit sage (Salvia gesneriiflora), South African sage (Salvia lanceolata), Graham's sage (Salvia microphylla), white sage (Salvia apiana), California sage (Salvia columbariae), autumn sage (Salvia greggii);
(v) at least one or more oregano products of: Greek oreganos (Origanum vulgare), Marjoram (Origanum majorana), Syrian oregano (Origanum syriacum), golden oregano (Origanum vulgare aureum), Italian oregano (Origanum×majoricum), Cuban oregano (Coleus amboinicus), Mexican oregano (Lippia graveolens), Vietnamese oregano (Plectranthus amboinicus);
(vi) ginseng (Panax quinquefolium);
(vii) black pepper (Piper nigrum; and
(viii) honey.

13. The pharmaceutical or nutritional composition of claim 12, wherein the composition comprises one to six parts ginger, one to six parts turmeric, one to six parts lemongrass, one to eight parts oregano, one to eight parts sage, one to eight parts ginseng, one to eight parts black pepper, and one to five parts honey.

14. A method of inhibiting and/or treating bacterial infection, viral infection, fungal infection, inflammation, chronic cough, neuroinflammation, depression, anxiety disorders, stress, or a combination thereof, the method comprising administering to a subject in need thereof an effective amount of the pharmaceutical or nutritional composition of claim 12.

15. A method of inhibiting and/or treating nerve degeneration associated with aging, the method comprising administering to a subject in need thereof an effective amount of the pharmaceutical or nutritional composition of claim 12.

16. A method of improving cognitive function, the method comprising administering to a subject in need thereof an effective amount of the pharmaceutical or nutritional composition of claim 12.

17. The method of claim 14, wherein the amount of the pharmaceutical composition administered is 450 mg/day to 25 g/day.

18. The method of claim 14, wherein the composition is orally administered.

19. The method of claim 14, wherein the composition is parenterally administered in one of the following ways: subcutaneously, intramuscularly, intravenously, or intrathecally.

20. A method of inhibiting and/or treating skin inflammation, acne, dark spots, wrinkles, or a combination thereof, the method comprising administering to a subject in need thereof an effective amount of the pharmaceutical or nutritional composition of claim 12 and wherein the administering is through topical administration and/or oral administration.
